(19) United States
(12) Patent Application Publication
(10) Pub. No.: US 2010/0104628 A1
(43) Pub. Date: Apr. 29, 2010

(54) METHOD OF TREATING NEUROBLASTOMA

(75) Inventors: Susan M. Ludeman, Durham, NC (US); Michael P. Gamcsik, Durham, NC (US); Timothy A. Driscoll, Durham, NC (US); James B. Springer, Durham, NC (US); O. Michael Colvin, Durham, NC (US); David J. Adams, Durham, NC (US); Karel Base, Durham, NC (US)

Correspondence Address: BANNER & WITCOFF, LTD., 1100 13th STREET, N.W., SUITE 1200, WASHINGTON, DC, 20005-4051, US

(73) Assignee: Duke University, Durham, NC (US)

(21) Appl. No.: 12/531,375

(22) PCT Filed: Mar. 28, 2008
(86) PCT No.: PCT/US08/04057

§ 371 Date: Sep. 15, 2009

Related U.S. Application Data
(60) Provisional application No. 60/907,322, filed on Mar. 28, 2007.

(51) Int. Cl. A61K 31/155; A61K 9/127; A61K 33/36; A61K 31/704; A61K 31/396; A61K 31/4375; A61K 31/216; A61P 35/00

(52) U.S. Cl. 424/450; 424/623; 514/34; 514/183; 514/283; 514/549; 514/634

(57) ABSTRACT

The present invention relates, in general, to neuroblastoma and, in particular, to a method of treating neuroblastoma tumors, including refractory neuroblastoma tumors. The invention also relates to compounds and compositions suitable for use in such a method.

This application claims priority from U.S. Provisional Application No. 60/907,322, filed Mar. 28, 2007, the entire content of which is hereby incorporated by reference.

This invention was made with government support under Grant Nos. RO1 CA16783, P30 CA14236 and U19 CA52857 awarded by the National Institutes of Health. The government has certain rights in the invention.

### TECHNICAL FIELD

The present invention relates, in general, to neuroblastoma and, in particular, to a method of treating neuroblastoma tumors, including refractory neuroblastoma tumors. The invention also relates to compounds and compositions suitable for use in such a method.

### BACKGROUND

Neuroblastoma (NB) is an extracranial, pediatric cancer derived from the sympathetic nervous system (Brodeur, Nat. Rev. Cancer 3:203-216 (2003)). It has been shown that 85-90% of both primary and metastatic NB tumors express the norepinephrine transporter (NET) (Carlin et al, Clin. Cancer Res. 9:3338-3344 (2003)). This transporter is currently used in conjunction with radiolabeled meta-iodobenzylguanidine (MIBG) both for therapeutic targeting of radiation and for imaging/diagnosis of NB (Matthay et al, J. Nucl. Med. 42:1713-1721 (2001); Montaldo et al, Int. J. Cancer 67:95-100 (1996); Tepmongkol et al, Med. Ped. Oncol. 32:427-431 (1999)).

Unlabeled MIBG has been shown to have anticancer activity both in vitro and in vivo (Ekelund et al, Biochem. Pharmacol. 61:1183-1193 (2001); Kuin et al, Brit. J. Cancer 79:793-801 (1999); Loesberg et al, Med. Ped. Oncol 35:793-798 (1991)). As with uptake, the extent of MIBG cytotoxicity strongly correlates with the level of NET expression (Matthay et al, J. Nucl. Med. 42:1713-1721 (2001); Montaldo et al, Int. J. Cancer 67:95-100 (1996); Cunningham et al, Med. Ped. Oncol. 35:708-711 (2000); Hopfner et al, Int. J. Cancer 101:210-216 (2002)).

NB cells are unusually sensitive to glutathione depletion (Anderson et al, Exp. Cell Res. 246:183-192 (1999)). More aggressive NB cell lines are especially sensitive to the glutathione-targeting agent, buthionine sulfoximine (BSO) (Anderson et al, Exp. Cell Res. 246:183-192 (1999)) and glutathione is known to play a role in resistance against many of the traditional drugs used as front-line therapies against NB (e.g., alkylating agents, platinating agents, camptothecins) (Colvin et al, Adv. Enzyme Regul. 33:19-26 (1993), Niimi et al, Cancer Res. 52:328-333 (1992)). Synergistic cytotoxicity has been shown for the combination of alkylating agents and glutathione-targeting agents, e.g., BSO and diallyl disulfide (Anderson et al, Exp. Cell Res. 246:183-192 (1999); Anderson et al, Med. Pediatr 35:659-662 (2000); Anderson et al, Bone Marrow Transplant. 30:135-140 (2002); Filomeni et al, Cancer Res. 63:5940-5949 (2003); Nicole et al, Biomed. Pharmacother. 52:349-355 (1998)).

It is the glutathione in the mitochondria that is proposed to play the most important role in determining the overall fate of any cell (Hall, Eur. J. Clin. Invest. 29:238-245 (1999); Meredith et al, Biochem Pharmacol. 32:1383-1388 (1983)). There is evidence that MIBG not only preferentially accumulates in NB cells but that it also concentrates in the mitochondria within NB (Gaze et al, Int. J. Cancer 47:875-880 (1991); Henry et al, Curr. Opin. Oncol. 17:19-23 (2005)).

To date, strategies for enhancing NB therapy that have focused on utilizing the NET mechanism of MIBG uptake or on depleting glutathione have not been used in combination. The present invention provides a unique approach to NB therapy that involves the use of single agents with moieties that exploit the NET-uptake mechanism as well as the sensitivity of cells to glutathione depletion in general, and mitochondrial glutathione depletion in particular. Viewing these diverse mechanisms of selectivity as parts of a composite target for single agent therapy allows for the design of therapeutics with significantly enhanced selectivity.

### SUMMARY OF THE INVENTION

The present invention relates, at least in part, to a method of treating neuroblastoma. In a preferred embodiment, the invention relates to a method of treating refractory neuroblastoma by administering an agent that selectively depletes glutathione within mitochondria of NET-expressing neuroblastoma. The invention further relates to agents suitable for use in such a method and to compositions comprising same.

Objects and advantages of the present invention will be clear from the description that follows.

### BRIEF DESCRIPTION OF THE DRAWINGS

FIG. 1. Meta-iodobenzylguanidine (MIBG) and general structure of proposed analogs.

FIG. 2. MIBG analogs targeted to glutathione.

FIG. 3. Synthesis of bis-Boc-4-CO₂CH₃-MIBG.

FIG. 4. Synthesis of modified MIBGs.

FIG. 5. MIBG-BSO analogs (n=0,1).

FIG. 6. MIBG-disulfides.

FIG. 7. MIBG-alkylating analogs.

FIG. 8. MIBG-(α,β-unsaturated carbonyls).

FIG. 9. Competitive NET binding assay.

FIGS. 10A-10C. Effects of MIBG and analogs on glutathione levels. (FIG. 10A) MIBG-disulfide dimer: cells treated with MIBG-disulfide dimer (5, 25 and 100 microM) for 1, 4 and 8 hrs. (FIG. 10B) 4-Chloromethyl-MIBG: glutathione levels in SK—N-BE(2c) treated with 25 microM 4-chloromethyl-MIBG over 4 hrs. (FIG. 10C) 4-Chloromethyl-MIBG and BSO: SK—N-BE(2c) (high NET) and MCF7 (no NET) treated with 5 microM 4-chloromethyl-MIBG, 25 microM BSO, or both.

### DETAILED DESCRIPTION OF THE INVENTION

NB is an extracranial tumor found in children. Patients with late stage and refractory disease have limited treatment options. The present invention expands these options by providing a method of treatment that comprises administering to patient in need thereof an agent that is highly selective for NB cells, particularly therapy-resistant cells, and that depletes glutathione within such cells. Agents suitable for such treatment methods can also be used as imaging agents and for therapeutic targeting of radiation (e.g., upon radioiodinization) to visualize and irradiate primary and metastatic tumors (e.g., neuroblastoma tumors) in vivo. While the methods of the invention (therapeutic and diagnostic (e.g., imaging)) are described in detail below with reference to neuroblastoma (including refractory neuroblastoma), these methods are generally applicable to tumors that overexpress the NET, including other neuroendocrine tumors such as some pancreatic tumors, pheochromocytomas, carcinoid tumors, paragangliomas, medullary carcinomas of the thyroid, chemodectomas, and other apudomas.

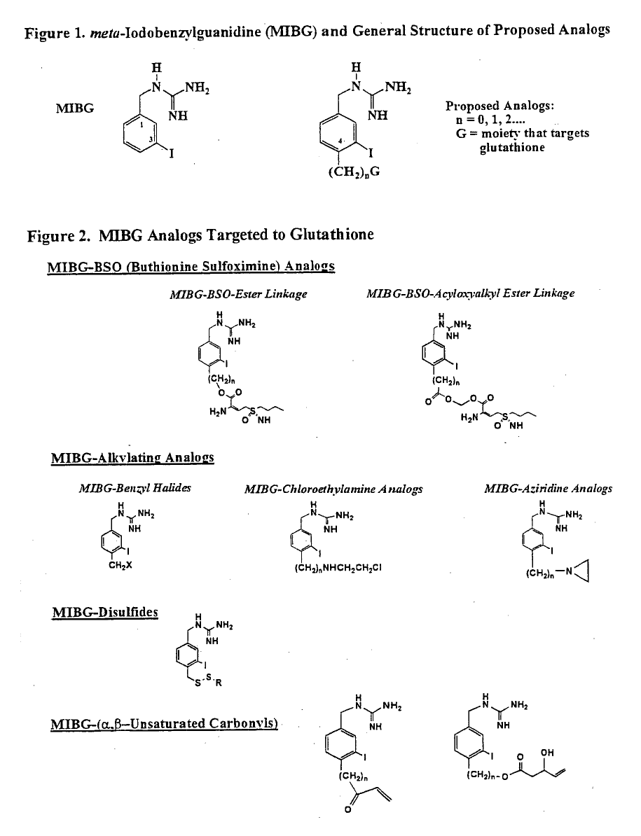

Agents preferred for use in the invention are taken up by NB cells via the NET, and target the glutathione metabolic pathway. In a preferred embodiment, the agents are glutathione-directed analogs of MIBG that effect selective glutathione depletion within the mitochondria of NET-expressing tumors. More specifically, agents appropriate for use in the methods described herein can incorporate aspects of targeted drug delivery by exploiting the specific NET-uptake properties of, for example, MIBG (FIG. 1).

Other compounds, including norepinephrine and related analogs/catecholamines, exhibit NET-uptake properties; indeed, NET is the target for many antidepressants, anesthetics and psychostimulants. Many of these compounds appear to function by binding to the NET and causing inhibition of or a change in its normal function rather than actually using transport properties. However, two compounds of note are naturally occurring 6-hydroxydopamine and its synthetic prodrug 6-fluorodopamine. The hydroxydopamine is cytotoxic to NB cells and depletes GSH (Tirmenstein et al, Toxicol. In Vitro 19:471-479 (2005)). The initial decrease in GSH, however, is followed by a substantial increase in GSH (cells adapt and ‘over-compensate’). 6-Fluorodopamine is NET-specific and is believed to convert intracellularly to 6-hydroxydopamine (Seitz et al, J. Neurochem. 75:511-520 (2000); Seitz et al, Med. Ped. One. 35:612-615 (2000)).

Modifications to the basic MIBG structure provide moieties that interfere with glutathione functions through various mechanisms of action (FIGS. 1 and 2). Preferred agents for use in the methods of the invention are of Formula I:

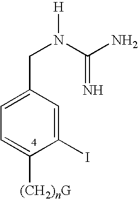

wherein n=0, or an integer from 1-10 (preferably, n=0, 1, 2, 3 or 4; more preferably, n=0, 1 or 2), and

G is a moiety that targets glutathione, and

pharmaceutically acceptable salts thereof.

Particularly preferred are the analogs set forth in FIG. 2 and the Example that follows,

wherein n=0 or 1,

X is a halogen, preferably, Br or Cl, and

R is an organic moiety, such as C₁₋₁₂ alkyl (e.g., C₁₋₄ alkyl), C₂₋₁₂ alkenyl (e.g., C₂₋₄ alkenyl), C₂₋₁₂ alkynyl (e.g., C₂₋₄ alkynyl), C₃₋₇ cycloalkyl, cyclic heteroalkyl, aryl, or heteroaryl; preferably, R is —CH₂CH═CH₂, —CH₂CH₂CH₃, —CH₂CH₃, —CO₂CH₃, or CH₂-MIBG, and

pharmaceutically acceptable salts thereof.

Agents of the invention, including those described in FIG. 2 and in the Example, can be prepared using processes known in the art and/or described in the following Example.

Several studies of NB have specifically investigated the effects of structural modifications at numerous positions throughout the MIBG molecule on NET-uptake (Ekelund et, Biochem. Pharmacol. 61:1183-1193 (2001), Hadrich et al, J. Med. Chem. 42:3101-3108 (1999), Smets et al, Cancer Chemother. Pharmacol. 21:9-13 (1988), Vaidynathan et al, Nucl. Med. Commun. 25:947-955 (2004), Vaidynathan et al, Appl. Radiat. Isot. 62:435-440 (2005), Vaidynathan et al, Bioorg. Med. Chem. 12:1649-1656 (2004), Vaidynathan et al, Bioconjugate Chem. 12:798-806 (2001), Vaidynathan et al, Eur. J. Nucl. Med. Mol. Imaging. 31:1362-1370 (2004), Vaidynathan et al, Bioconjugate Chem. 9:758-764 (1998), Van Den Berg et al, Cancer Chemother. Pharmacol. 40:131-137 (1997)). The results of these studies demonstrate that only C-4 can be substantially modified without significant loss of affinity for NET.

The invention further relates to compositions comprising agents suitable for use in the invention formulated with an appropriate carrier. The MIBG analogs can be formulated with the standard chemotherapeutics and administered simultaneously or they can be formulated and administered separately (e.g., the MIBG analog can be administered first followed by the chemotherapeutic, or visa versa). Formulation techniques known in the art can be used, for example, as described in Remington's Pharmaceutical Sciences, 17th ed., Mack Publishing Company, Easton, Pa., (1985). The composition can be present, for example, as a solution (e.g., a sterile solution) or suspension. The composition can also be present in dosage unit form (e.g., as a tablet or capsule). The nature of the formulation can vary depending, for example, on the agent and on the route of administration (e.g., intravenous).

Representative delivery regimens include, without limitation, oral or parenteral intravenous infusion.

In general, the agents of the invention (e.g., MIBG analogs), or pharmaceutically acceptable salts thereof, can be administered in amounts between about 1 microgram and 200 milligrams per kg body weight per day, preferably, from about 1 microgram to about 10 milligrams per kg body weight per day. Dosages can be delivered by a single administration, by multiple applications, by continuous intravenous infusion, or via controlled release, as needed to achieve the results sought.

Optimum dosing regimens can be readily determined by one skilled in the art and can vary with the agent, the patient and the effect sought.

The biodistribution of the MIBG-analogs can be shifted toward selected accumulation in NB through MIBG pretreatment and/or liposomal encapsulation.

MIBG shows at least transient accumulation in the liver, lung, bladder, and heart. It has been shown in both mice and clinical settings that NB tumor to non-tumor ratios of ¹³¹I-MIBG concentrations can be increased through pre-treatment with unlabeled MIBG (Taal et al, Ann. Oncol. 11:1437-1443 (2000), Hoefnagel et al, Nucl. Med. Commun. 21:755-761 (2000)). Pre-treatment with unlabeled MIBG provides a strategy whereby tumor targeting of MIBG-analogs can be enhanced.

NB cells are highly vascularized and this can be used to advantage in improving localized drug delivery. Tumor vasculature is more permeable than that of normal tissue and particulate drug carriers, such as liposomes, localize in the interstitial space of tumors through the enhanced permeability and retention (EPR) effect (Allen et al, Science 303:1818-1822 (2004)). This passive targeting is inherent in the design of pegylated liposomes which have been shown to be effective in enhancing drug delivery to NB (Michaelis et al, Oncol. Rep. 13:157-160 (2005): Nagae et al, J. Pediatr. Surg., 33:1521-1525 (1998)). There are many methods by which drugs can be incorporated into liposomes and many of these are applicable to a wide range of drugs (Michaelis et al, Oncol. Rep., 13:157-160 (2005); Pastorino et al, Cancer Res, 63:7400-7409 (2003); Pastorino et al, Cancer Res, 63:86-92 (2003); Sadzuka et al, J Control Release, 108:453-459 (2005); Viglianti et al, Mag. Res. Med., 51:1153-1162 (2004)). The low extracellular pH of tumor cells hydrolyzes the liposomes and the drug is released. This passive targeting has been shown to be effective in enhancing drug delivery to many cancers, including NB (Allen et al, Science 303:1818-1822 (2004), Michaelis et al, Oncol. Rep. 13:157-160 (2005), Nagae et al, J. Pediatr. Surg. 33:1521-1525 (1998)). Thus, through liposomal encapsulation, MIBG-analogs can be released more selectively to the interstitial space of NB cells where cellular uptake is then facilitated by NET.

Agents described herein are, advantageously, used in combination with traditional chemotherapeutics, preferably, those that have cytotoxicities dependent on glutathione levels, such as alkylating agents (e.g., melphalan, ifosfamide, cyclophosphamide), platinating agents (e.g., carboplatin, cisplatin), camptothecins (e.g., topotecan, irinotecan), arsenic trioxides, and doxorubicin. Such combination therapy is particularly well suited to the treatment of refractory neuroblastoma tumors. Co-administration or rapid sequencing of agents of the invention with traditional NB therapeutics can enhance the efficacy of the traditional chemotherapeutics and either reduce the required dose or make the standard dose more effective.

Certain aspects of the invention are described in greater detail in the non-limiting Example that follows. (See also DE 19817517; Vaidyanathan et al, Bioconj. Chem. 12:428-438 (2001); Vaidyanathan et al, Bioconj. Chem. 12:798-806 (2001); U.S. Pat. No. 4,622,217; Vaidyanathan et al, Bioorg. Med. Chem. 12:1649-1655 (2004).)

### Example

### Synthesis of MIBG-Analogs Targeted to Glutathione

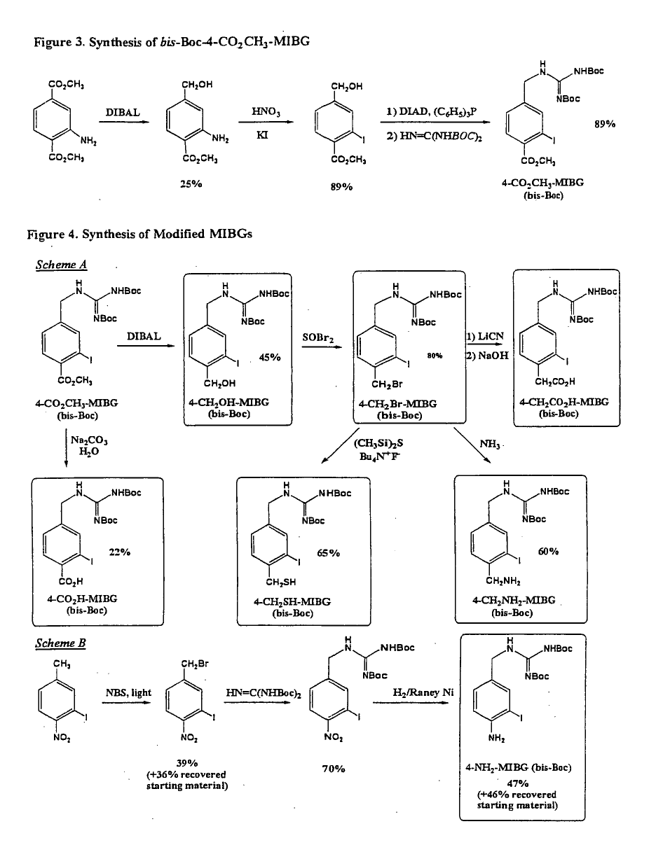

### Synthesis of Modified MIBG Precursors (FIGS. 3 and 4)

The MIBG-analogs require a modified MIBG to be linked to a moiety that targets glutathione. The protected bis-Boc-4-CO₂—CH₃-MIBG can act as a focal compound that provides facile entry to other requisite modified MIBG precursors (FIG. 3). FIG. 4 illustrates the use of bis-Boc-4-CO₂CH₃-MIBG to generate other C-4 modified MIBGs. The synthesis of bis-Boc-4-OH-MIBG is not shown but has been reported (Vaidynathan et al, Bioconjugate Chem. 12:786-797 (2001)).

### Synthesis of MIBG-BSO Analogs as Enzyme Inhibitors (FIG. 5)

The important role of glutathione in therapy response in cancer has led to the investigation of agents that can modify glutathione levels to enhance therapy. One such agent is buthionine sulfoximine (BSO). It is extremely selective for glutathione because it inhibits a key enzyme in glutathione biosynthesis (Griffith, O. W., Glutathione Centennial Molecular Perspectives and Clinical Implications. Meister, A. (Ed.), New York, Academic Press, 285-299 (1989)). This observation has led to the inclusion of BSO in NB treatments and clinical trials The drawback to the use of systemic BSO is that glutathione will also be depleted in normal tissue and, when used in combination with cytotoxic drugs, can lead to increased systemic toxicity. BSO dramatically increases the toxicity of many anticancer drugs in resistant cancers (Ozols, Seminars in Oncology XII(3, Suppl 4):7-11 (1985)). In NB, BSO potentiates melphalan toxicity in refractory tumors (Anderson et al, Bone Marrow Transplant. 30:135-140 (2002)).

The MIBG-BSO conjugates are designed to release free BSO which then inhibits glutathione synthesis thereby resulting in depletion of glutathione stores via normal cellular catabolism. This process of glutathione depletion is very slow in cardiac tissue; therefore, an MBIG-BSO conjugate would likely have minimal effect on glutathione levels in heart or other tissues where glutathione turnover is low.

In this set of analogs, the MIBG moiety acts as the carrier that will be bonded to BSO through a simple ester or an acyloxyalkyl ester linkage. Esterases are ubiquitous in vivo and there is the possibility of ‘premature’ (extracellular) scission of the bond between the MIBG carrier and BSO when they are linked by a conventional ester moiety. This type of analog can be protected in vivo by liposomal delivery. Evidence has been reported that acyloxyalkyl esters survive extracellular hydrolysis (Nudelman et al, J. Med. Chem. 43:2962-2966 (2000)).

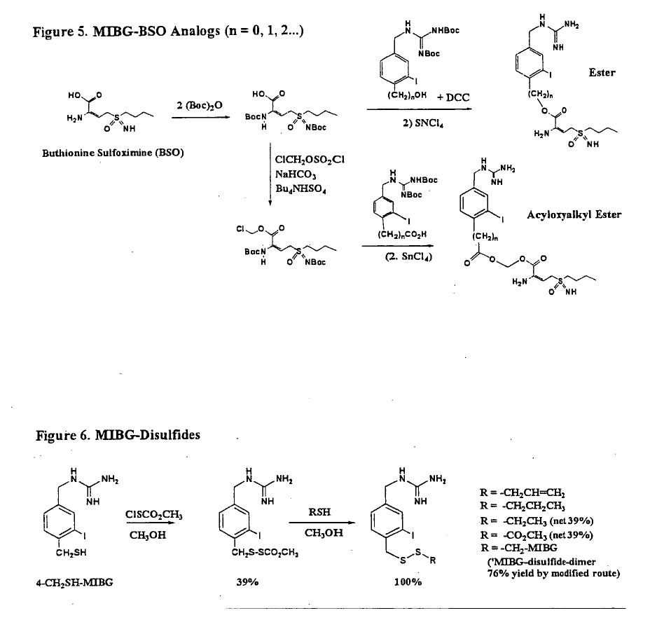

### Synthesis of MIBG-S—S—R Analogs for Disulfide-Thiol Exchange (FIG. 6)

A disulfide-thiol exchange is generally reversible but under the conditions of high glutathione (GSH) concentration, the reaction will be shifted to the right (as shown below):

MIBG-S—S—R+GSH→MIBG-S—S-G+HSR

The synthesis of mixed disulfides as shown in FIG. 6 is based on published procedures (Brois et al, J. Am. Chem. Soc. 92:7629-7631 (1970)). One analog is based on diallyl disulfide [(—SCH₂CH═CH₂)₂], a compound that induces apoptosis in human NB SH—SY5Y (Filomeni et al, Cancer Res. 63:5940-5949 (2003)).

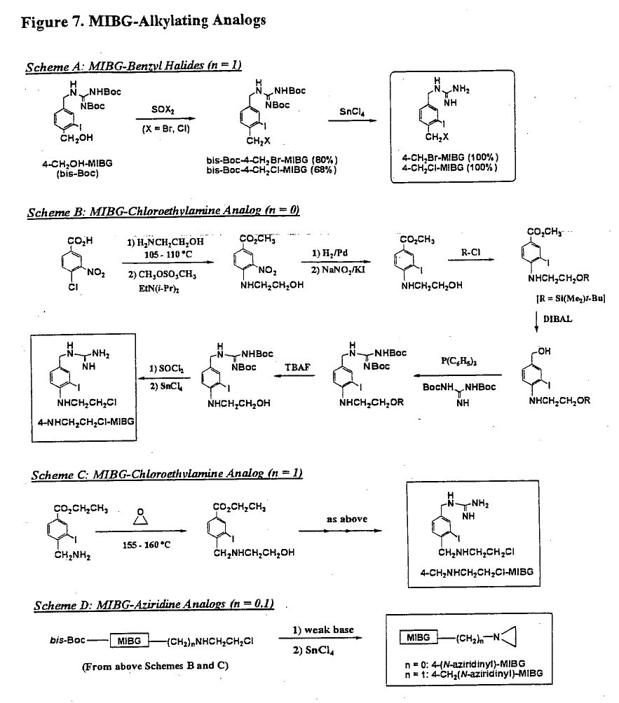

### Synthesis of MIBG-Alkylating Analogs for Substitution Reactions (FIG. 7)

Direct reaction between glutathione (GSH) and the MIBG analogs which are alkylating agents represents a third mechanistic design: glutathione depletion by an irreversible, nucleophilic substitution reaction.

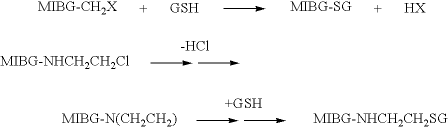

The pathway shown in Scheme B of FIG. 7 was carried out using HN(CH₂CH₂Cl)₂ in place of H₂NCH₂CH₂Cl as a starting material. The yields for the steps in the pathway ranged from 49% to 100%.

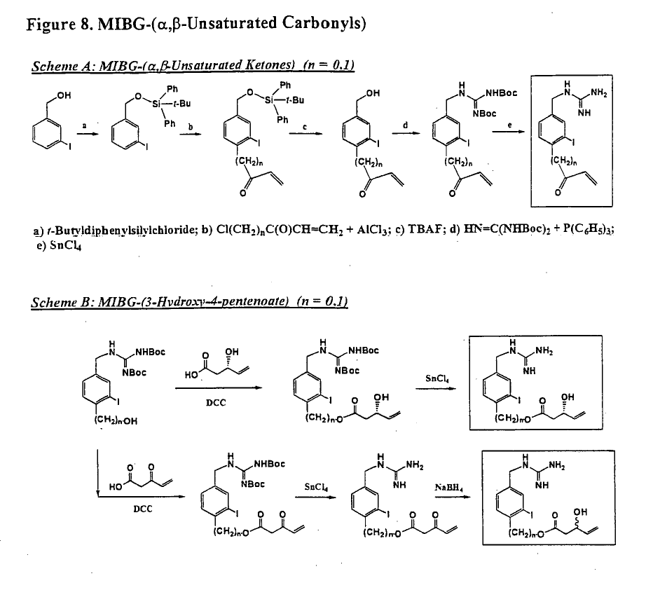

### Synthesis of MIBG-C(O)CH═CH₂ Analogs for Conjugate Addition Reactions (FIG. 8)

O═C(R)—CH═CH₂+GSH→O═C(R)—CH₂—CH₂—SG

The analog in Scheme A of FIG. 8 will react directly with glutathione; those in Scheme B of FIG. 8 are designed as prodrugs, similar to the MIBG-BSO analog wherein the MIBG moiety acts as carrier and the drug must be released hydrolytically to achieve activity. 3-Hydroxy-4-pentenoate [CO₂H—CH₂CH(OH)CH═CH₂] is known to oxidize selectively in mitochondria; the product of this reaction [CO₂H—CH₂C(O)CH═CH₂] then reacts rapidly and irreversibly with mitochondrial glutathione (Hashmi et al, Chem. Res. Toxicol. 9:361-364 (1996); Shan et al, Chem. Death. Chem. Res. Toxicol. 6(1):75-81 (1993)). While the mitochondrial pool of glutathione is relatively small in comparison with that of the cytosol, it is mitochondrial glutathione that is believed to play a significant role in protecting cells from damaging xenobiotics.

As discussed above for the MIBG-BSO analog, esterases are ubiquitous in vivo and there is the possibility of ‘premature’ (extracellular) scission of the ester bond between the MIBG carrier and the hydroxypentanoate. The same protective measures can be used for this analog as were described for the MIBG-BSO analog (i.e., liposomal delivery and linkage of drug to MIBG by the alternative acyloxyalkyl ester bond).

### Results

### Determination of Reaction Kinetics (Table 1)

Proton NMR was used to determine the half-lives for chloro- and bromomethyl-MIBG (Table 1). These analogs react with nucleophiles via an S_{N}2 mechanism; therefore, the rate of the reaction depends on the strength and concentration of the nucleophile as well as the nature of the leaving group. Thus, the half-lives of the analogs are predictably longer in buffer than they are in buffer with added glutathione (a relatively strong nucleophile) and, under the same conditions, the bromo analog (better leaving group) reacts faster than the chloro analog.

TABLE 1
Stability of MIGB-Analogs, 37° C., pH 7.4^{a}

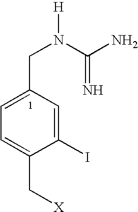

X = Br; Bromomethyl-MIBG
X = Cl; Chloromethyl-MIBG

| | | Half-life w/10 |
|---|---|---|
| Analog | Half-life | eq GSH |
| Bromomethyl-MIBG | 42.5 ± 0.7 min | <5 sec |
| Chloromethyl-MIBG | 580 ± 25 min | 50.6 ± 3.2 sec |

^{a}BisTris buffer, 5% DMSO (to improve solubility for NMR).
Note:
DMSO may slow reaction rate relative to solution without DMSO.

The results show that both analogs react rapidly with glutathione

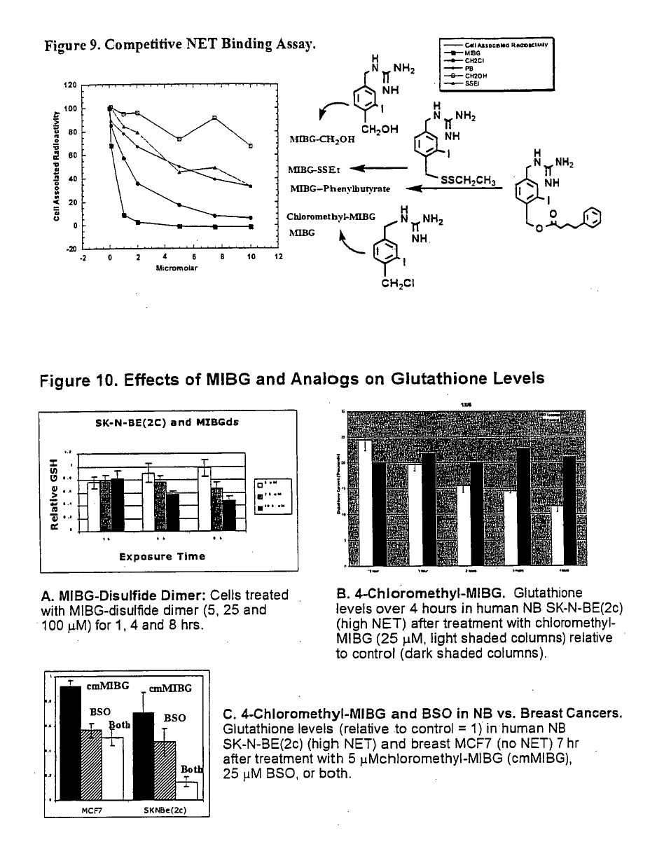

### NET Competitive Binding Assay (FIG. 9)

An assay to assess binding of various MIBG-analogs to the NET was performed by published methods (Vaidynathan et al, Nucl. Med. Commun. 25:947-955 (2004), Vaidynathan et al, Bioorg. Med. Chem. 12:1649-1656 (2004), Vaidynathan et al, Bioconjugate Chem. 12:798-806 (2001), Vaidynathan et al, Eur. J. Nucl. Med. Mol. Imaging 31:1362-1370 (2004)). In brief, neuroblastoma SK—N—SH cells (moderate levels of NET) were treated with [¹²⁵I]MIBG and with increasing concentrations of unlabeled MIBG or MIBG-analogs. At the end of a 2 hr incubation and appropriate work-up, the cell-bound radioactivity was counted. Total cell-bound radioactivity minus non-specific cell-bound radioactivity gave a value for specific binding which was plotted in FIG. 9 against the concentration of test compounds. At 5 μM, cold MIBG displaced “100%” of the labeled-MIBG (i.e., “zero” cell-bound radioactivity). At 5 μM, chloromethyl-MIBG displaced 83%; the disulfide MIBG-SS-ethyl and the ester MIBG-phenylbutyrate displaced 50-55%; and hydroxymethyl-MIBG displaced 27%.

These results show that chloromethyl-MIBG binds quite well to the NET; conversely, its hydrolysis product, hydroxymethyl-MIBG, does not. Due to the fact that the NET-affinities for these compounds are so different, it can be concluded that the chloromethyl-MIBG did not hydrolyze to any significant extent under the conditions of the binding assay. This provides confidence that the data reflects NET-binding of the actual chloromethyl-MIBG and not a degradation product. Additional evidence to this effect is given in the cytotoxicity studies described below. The binding results for MIBG-phenylbutyrate (included in this assay for its size) and MIBG-S—S—CH₂CH₃ suggest that NET can accommodate a range of molecular sizes.

### Glutathione Depletion In Vitro (FIG. 10)

The NB cell line SK—N-BE(2c) (high net) was treated with MIBG, MIBG-disulfide dimer (MIGB-S—S-MIBG), chloromethyl-MIBG (cmMIBG), and BSO. For comparison, the human breast cancer line MCF7 (no NET) was treated with chloromethyl-MIBG and BSO. At specific times, intracellular glutathione in drug-treated cells was determined relative to that in untreated cells (control).

MIBG alone did not significantly affect glutathione levels at concentrations up to 25 μM and incubation times up to 4 hrs (data not shown).

With 5 μM MIBG-disulfide dimer (see FIG. 6 for chemical structure), a transient loss of glutathione was observed but within 8 hrs the level of glutathione returned to pre-treatment levels. At higher concentrations of the disulfide, there was an extensive loss of glutathione and cell viability with no recovery (FIG. 10A).

SK—N-BE(2c) (high NET) cells were treated with a single concentration of chloromethyl-MIBG (25 μM at t=0). Glutathione concentrations in treated and untreated cells were measured at t=0.5, 1, 2, 3 and 4 hrs. Glutathione levels in drug-treated cells progressively fell, with a concentration 50% that of control at t 4 hrs (FIG. 10B).

Further evidence for the selectivity of chloromethyl-MIBG and NET as well as glutathione-targeting is demonstrated in studies of glutathione depletion in MCF7 (no NET) and SK—N—Be(2c) (high NET) cells (FIG. 10C). Cells were exposed to 5 μM chloromethyl-MIBG (cmMIBG), 25 μM BSO and a combination of 5 μM chloromethyl-MIBG+25 μM BSO (both) for 7 hrs. Compared to controls, chloromethyl-MIBG alone depleted glutathione by about 30% in SK—N—Be(2c) cells and 8% in MCF7. BSO, as expected, depleted MCF7 and SK—N—Be(2c) by similar amounts (42 and 54% of controls, respectively). In combination studies, chloromethyl-MIBG+BSO depleted levels by about 85% in the SK—N—Be(2c) cells and had much less effect on the MCF7 cells with glutathione reduced by 49% relative to controls. These data for MCF7 vs. SK—N—Be(2c) are consistent with a NET-dependent uptake of chloromethyl-MIBG. As expected, no NET-dependency was observed for BSO.

### Cytotoxicity Data Related to Chloromethyl-MIBG (Table 2)

Preliminary toxicity testing has concentrated on chloromethyl-MIBG because this analog has shown the strongest binding to NET (FIG. 9). As determined from IC₅₀ values, this compound (administered alone) is most toxic for cells with high levels of NET-expression (Table 2B). In addition, the toxicity of chloromethyl-MIBG is significantly higher than that of either MIBG (no glutathione-targeting moiety, Table 2A) or structural analog benzylchloride (C₆H₅CH₂Cl; no NET-targeting moiety, Table 2C).

Melphalan is a front-line treatment for neuroblastoma and is in clinical trial as part of a new combination therapy for NB with the non-selective, glutathione-targeting agent BSO. Melphalan does show good activity against the NB cell line as compared to that against the MCF7 breast cancer line (Table 2D). However, in the presence of 5 μM chloromethyl-MIBG, the IC₅₀ of melphalan against SK—N—BE(2c) (high NET) was enhanced while that of MCF7 (no NET) was unchanged (Table 2D). MIBG did not potentiate the activity of melphalan in either cell line (Table 2D).

To provide additional evidence that chloromethyl-MIBG is taken up via NET, a competition experiment was performed by adding MIBG to the cytotoxicity assay (Table 2E). In side-by-side experiments, SK—N-BE(2c) cells were treated with:

(a) melphalan (0.4-400 μM) plus 5 μM chloromethyl-MIBG plus 5 μM MIGB; or

(b) melphalan (0.4-400 μM) plus 5 μM chloromethyl-MIBG plus vehicle.

The IC₅₀ for melphalan in the melphalan/chloromethyl-MIBG combination was decreased in the presence of MIBG (i.e., 8.1 without vs. 22.9 μM with MIBG, Table 2E). Since MIBG alone has no effect on the IC₅₀ of melphalan, it is reasonable to assume that MIBG is decreasing the activity of chloromethyl-MIBG. Decreased activity of chloromethyl-MIBG would result from less efficient cellular uptake. Less efficient uptake of chloromethyl-MIBG would result from competition between that compound and MIBG for NET.

In another experiment, the IC₅₀ values for 4-chloromethyl-MIBG were determined in the presence and absence of desipramine, a selective inhibitor of NET (Table 2F) (Zhu et al, J. Neurochem. 82:146-153 (2002)). Against NET-positive neuroblastoma SK—N-BE(2c), the presence of desipramine resulted in a nearly 5-fold increase in the IC₅₀ value for chloromethyl-MIBG. Against NET-negative breast cancer MCF7, desipramine had no impact on the IC₅₀ value for chloromethyl-MIBG. This data provided strong evidence for the validity of a key drug design factor, i.e., direct dependence of cytotoxicity on NET-uptake.

TABLE 2
Cytotoxicity (IC₅₀, μM) Determinations^{a} (24 hr assay)

| | | | | |
|---|---|---|---|---|
| | SK-N-BE(2c) | SK-N-MC | MCF7 | IMR32 |
| compound | (NET ++++) | (NET ++) | (NET 0) | (NET 0/+) |

A. Toxicity MIBG

| | | |
|---|---|---|
| MIBG | 200 ± 12 | 232 ± 13 |

B. Toxicity chloromethylMIBG

| | | | | |
|---|---|---|---|---|
| cmMIBG (1% EtOH) | 5 | 25 | 50 | 63 |
| cmMIBG | 5.7 ± 1.5 | | 56.9 ± 6.2 | |

C. Toxicity chloromethyl-benzene

| | | |
|---|---|---|
| chloromethyl-benzene | 172 ± 31 | 390 ± 77 |

D. Toxicity Melphalan, MIBG, chlormethylMIBG (cmMIBG)

| | | |
|---|---|---|
| Melphalan alone | 36.2 ± 5.2 | 250 ± 112 |
| MIBG alone | 167 ± 5 | 227 ± 9 |
| Melphalan + 5 μM MIBG | 43.5 ± 7.0 | 386 ± 181 |
| Melphalan + 5 μM cmMIBG | 11.3 ± 5.4 | 276 ± 28 |

E. Competition Experiment

| | |
|---|---|
| Melphalan + 5 μM cmMIBG | 8.1 ± 5.4 |
| Melphalan + 5 μM cmMIBG + | 22.9 ± 2.7 |
| 5 μM MIBG | |

F. NET Inhibition with desipramine (48 hr assay)^{b}

| | | |
|---|---|---|
| cmMIBG | 3.2 ± 0.6 | 19.8 ± 1.3 |
| cmMIBG + 1.5 μM desipramine | 14.3 ± 1.7 | 20.2 ± 1.9 |

^{a}Cells were exposed to drugs for 24 h then viability assessed by MTT. Drugs dissolved in DMSO unless otherwise noted (final DMSO concn 1%)
^{b}Cells were treated for 40 min with 1.5 micromolar desipramine and then co-incubated with varying concentrations of chloromethyl-MIBG for 24 h followed by 24 h recovery time and then MTT assay of cell viability. cmMIBG dissolved in DMSO (final DMSO concn 1%).

### Cytotoxicity Data of Other MIBG Analogs (Table 3)

MIBG-S-glutathione, which is given by the reactions of MIBG-benzyl halides (FIG. 7A) with glutathione, was synthesized in order to determine if any toxicity is associated with the formation of this product. An aqueous solution of glutathione was added to a DMSO solution of 4-chloromethyl-MIBG hydrochloride (1.5 equiv). The pH was adjusted to 7.4 and the mixture was heated overnight at 42° C. Filtration and lyophilization gave a white powder which was identified by NMR as MIBG-S-glutathione. This compound gave relatively high IC₅₀ values against neuroblastoma SK—N-BE(2c) and breast cancer MCF7 thereby suggesting that this material would be a non-toxic by-product of treatment with the MIBG-benzyl halide analogs (Table 3).

As an example of the analogs shown in FIG. 6, MIBG-ethyl-disulfide was synthesized (FIG. 6, R═CH₂CH₃) and tested in vitro. MIBG-ethyl-disulfide exhibited cytotoxicity comparable to or better than melphalan; however, it was unclear if NET-dependent uptake was operative since IC₅₀ values were similar against NET-positive NB SK—N-BE(2c) and NET-negative breast cancer MCF7 (Table 3).

TABLE 3
Cytotoxicity (IC₅₀, μM) Determinations^{a} (48 hr assay)

| Compound | | | |
|---|---|---|---|
| (dissolved in 1% | | | |
| DMSO unless noted | SK-N-BE(2c)^{b} | SK-N-SH^{b} | MCF7^{b} |
| otherwise) | (NET ++++) | (NET ++) | (NET −) |
| Chloromethyl-MIBG | 4.63 ± 1.58 (n = 3) | 18.35 ± 9.10 (n = 5) | 14.33 ± 2.13 (n = 3) |
| MIBG-S-Glutathione | 390 ± 475 (n = 13) | nd^{c} | >>1 × 10³ (n = 5) |
| MIBG-Ethyl-Disulfide | 27.35 ± 4.09 (n = 4) | nd^{c} | 24.21 ± 7.21 (n = 3) |
| MIBG | 142 ± 21 (n = 8) | 190 ± 22 (n = 8) | 137 ± 27 (n = 8) |
| Melphalan (L-PAM) | 25.81 ± 3.54 (n = 6) | 54.14 ± 13.76 (n = 5) | 126 ± 28 (n = 4) |

^{a}Standard cytotoxicity assay with 24 h drug exposure followed by 24 h recovery time and then MTT assay of cell viability.
^{b}n = Number of repeat experiments.
^{c}nd = Not determined.

### Effect of Chloromethyl-MIBG on the Cytotoxicity of Melphalan (Table 4).

Expanding on the combination study reported in Table 2D, changes in the cytotoxicity (IC₅₀) of melphalan were determined as a consequence of varying chloromethyl-MIBG concentrations and exposure times. A dose dependent effect was observed: 5 μM chloromethyl-MIBG increased the potency of melphalan more than did 1 μM chloromethyl-MIBG. This effect was observed in all cell lines studied but it was most pronounced in the NB line with the highest NET activity.

As shown in FIG. 10B, treatment of NB SK—N-BE(2c) cells with chloromethyl-MIBG caused intracellular concentrations of glutathione to progressively decrease over time. This suggested that optimal sensitivity of cells to melphalan might occur some hours after chloromethyl-MIBG treatment. Thus, cells were pre-treated for 3 hrs with 1 or 5 μM chloromethyl-MIBG before the addition of melphalan. For the NB cell lines, the melphalan IC₅₀ values were relatively insensitive to the timing of chloromethyl-MIBG addition.

TABLE 4
Effects of Chloromethyl-MIBG on the Cytotoxicity (IC₅₀, μM) of Melphalan^{a}
(48 hr assay)

| Drug(s) | | | |
|---|---|---|---|
| (dissolved in 1% DMSO | SK-N-BE(2c)^{b} | SK-N-SH^{b} | MCF7^{b} |
| unless noted otherwise) | (NET ++++) | (NET ++) | (NET −) |
| Melphalan (L-PAM) | 25.8 ± 3.5 (n = 6) | 54.1 ± 13.8 (n = 5) | 126 ± 28 (n = 4) |
| Chloromethyl-MIBG (cmMIBG) | 4.6 ± 1.6 (n = 3) | 18.4 ± 9.1 (n = 5) | 14.3 ± 2.1 (n = 3) |
| Melphalan + 1 μM cmMIBG | 13.9 ± 4.7 (n = 5) | 36.7 ± 18.7 (n = 4) | 127 ± 17 (n = 3) |
| (added to cells at same time) | | | |
| Melphalan + 5 μM cmMIBG | 7.3 ± 1.3 (n = 3) | 29.9 ± 10.6 (n = 4) | 75 ± 25 (n = 3) |
| (added to cells at same time) | | | |
| 1) 1 μM cmMIBG (3 hrs) | 14.0 ± 3.7 (n = 3) | 29.8 ± 4.8 (n = 5) | 60 ± 7 (n = 4) |
| 2) Melphalan | | | |
| (cells treated with cmMIBG for 3 hrs | | | |
| and then melphalan added) | | | |
| 1) 5 μM cmMIBG (3 hrs) | 10.2 ± 0.9 (n = 2) | 38.4 ± 7.7 (n = 4) | 43 ± 11 (n = 4) |
| 2) Melphalan | | | |
| (cells treated with cmMIBG for 3 hrs | | | |
| and then melphalan added) | | | |

^{a}Standard cytotoxicity assay with 24 h drug exposure followed by 24 h recovery time and then MTT assay of cell viability.
^{b}n = Number of repeat experiments.

### Study of Maximum Tolerated Dose (MTD) in Mice for Chloromethyl-MIBG.

The MTD study utilized 4 groups of 5 HRLN female nu/nu mice. Mice in each group were injected i.v. with a single dose of chloromethyl-MIBG in 1% DMSO in water [5, 10, 15, or 20 mg/kg (drug/animal)]. Drug solutions were made fresh at room temperature and injections were completed within 20 minutes of making the drug solution (under these conditions, the half-life of chloromethyl-MIBG was estimated to be >10 hrs). The dosing volume was 0.20 mL/20 g mouse (adjusted accordingly for body weight). Animals were followed for 21 days post-injection; body weight was monitored daily for 5 days and then biweekly. The “endpoint MTD” was defined as a mean weight loss of >20% or death of >10% of animals in a group.

Upon drug injection, the groups dosed at 10, 15 and 20 mg/kg exhibited signs of hypoactivity, labored breathing, darkening of skin color, and/or seizures/tremors. All adverse symptoms went away within 30 seconds. Such discomfort at injection may be mitigated in the future by slowing the rate of drug infusion. Over the course of the study no other adverse events were reported and there were no animal deaths. The mean weight changes for the 4 groups at Day 21 varied between +2 and +12%.

This experiment demonstrated that the MTD for chloromethyl-MIBG in mice is >20 mg/kg. For comparison, the MTD for melphalan in mice has been reported at 10-15 mg/kg with a 20 mg/kg single injection causing a 100% death rate by one week [(101×C3H)F₁ male mice, single injection, i.p.] (Russell et al., Mut. Res. 282:151-158 (1992)). Higher MTD values are desirable because they are associated with less dose-limiting toxicity. (Piedmont Research Center, Morrisville, N.C., was contracted to carry out the MTD study).

Summarizing, in studies against 4 cell lines, chloromethyl-MIBG cytotoxicity correlated with levels of NET expression. This was consistent with the competitive binding assay which showed that the NET-affinity of chloromethyl-MIBG was comparable to that of MIBG. It must be emphasized that these data are for a compound that was designed to be an agent that only interferes with glutathione metabolism. As such, chloromethyl-MIBG was not specifically envisioned as a stand-alone cytotoxic but rather as an agent that would enhance the efficacy of chemotherapeutics, such as melphalan, that are known to be affected by glutathione levels in the cell. In fact, as designed, chloromethyl-MIBG markedly potentiated the cytotoxicity of melphalan against an NB cell line [SK—N-BE(2c)] that is known to exhibit significant multi-drug resistance. This potentiation was somewhat reversed in the presence of MIBG (a competitor for NET binding), suggesting that chloromethyl-MIBG activity occurs via NET. This hypothesis was further supported by the finding that chlormethyl-MIBG was significantly less toxic against NB cells that had been pre-treated with the selective NET-inhibitor desipramine. These results support the concept that glutathione-directed MIBG-analogs will limit non-specific depletion of glutathione by targeting NET-expressing tissue and, therefore, will more efficiently enhance the efficacy of traditional NB chemotherapeutics through drug combination treatments.

All documents and other information sources cited above are hereby incorporated in their entirety by reference.

What is claimed is:

1. A method of treating a tumor comprising administering to a patient in need of such treatment an agent that selectively depletes glutathione within mitochondria of tumor cells that express norepinephrine transporter (NET), wherein said agent is administered in an amount sufficient to effect said treatment.

2. The method according to claim 1 wherein said tumor is a neuroblastoma.

3. The method according to claim 2 wherein said neuroblastoma is a refractory neuroblastoma.

4. The method according to claim 1 wherein said tumor is a pancreatic tumor, pheochromocytoma, carcinoid tumor, paraganglioma, medullary carcinoma of the thyroid, or chemodectoma.

5. The method according to claim 1 wherein said agent is an analog of meta-iodobenzylguanidine (MIBG) that selectively depletes glutathione within mitochondria of NET-expressing tumor cells.

6. The method according to claim 1 wherein said agent is of Formula I 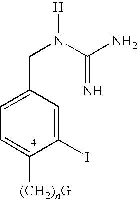
wherein n=0, or an integer from 1-10, and G is a moiety that targets glutathione, or pharmaceutically acceptable salt thereof.

7. The method according to claim 6 wherein n is 0, 1 or 2.

8. The method according to claim 6 wherein said compound is 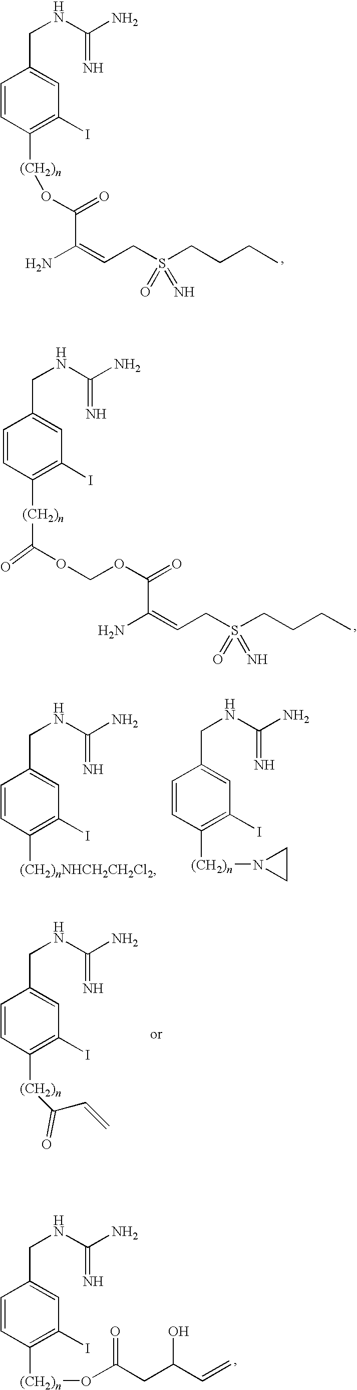
wherein n=0 or 1.

9. The method according to claim 6 wherein said compound is 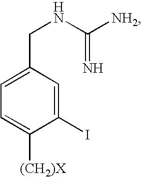
wherein X is a halogen.

10. The compound according to claim 6 wherein said compound is 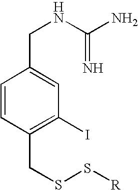
wherein R is C₁₋₁₂alkyl, C₂₋₁₂alkenyl, C₂₋₁₂alkyl, C₃₋₇cycloalkyl, cyclic heteroalkyl, aryl or heteroaryl.

11. The method according to claim 10 wherein R is C₁₋₄alkyl, C₂₋₄alkenyl or C₂₋₄alkynyl.

12. The method according to claim 10 wherein R is —CH₂CH═CH₂, —CH₂CH₂CH₃, —CH₂CH₃, —CO₂CH₃ or —CH₂-MIBG.

13. The method according to claim 1 wherein said agent is encapsulated in a liposome.

14. The method according to claim 1 wherein meta-iodobenzylguanidine is administered to said patient prior to administration of said agent.

15. The method according to claim 1 wherein said method further comprises administering to said patient an alkylating agent, platinating agent, camptothecin, arsenic trioxide or doxorubicin.

16. A compound of Formula I 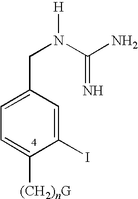
wherein n=0, or an integer from 1-10, and G is a moiety that targets glutathione, or pharmaceutically acceptable salt thereof.

17. The compound according to claim 16 wherein n is 0, 1 or 2.

18. The compound according to claim 16 wherein said compound is 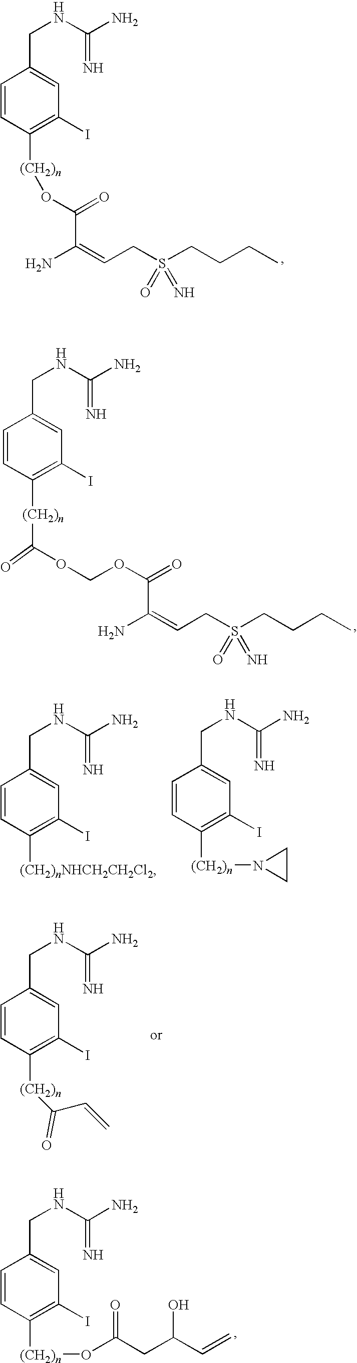
wherein n=0 or 1.

19. The compound according to claim 16 wherein said compound is 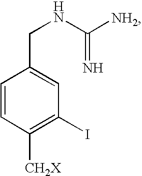
wherein X is a halogen.

20. The compound according to claim 16 wherein said compound is 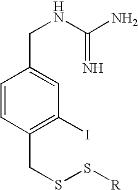
wherein R is C₁₋₁₂alkyl, C₂₋₁₂alkenyl, C₂₋₁₂alkyl, C₃₋₇cycloalkyl, cyclic heteroalkyl, aryl or heteroaryl.

21. The compound according to claim 20 wherein R is C₁₋₄alkyl, C₂₋₄alkenyl or C₂₋₄alkynyl.

22. The compound according to claim 20 wherein R is —CH₂CH═CH₂, —CH₂CH₂CH₃, —CH₂CH₃, —CO₂CH₃ or —CH₂-MIBG.

23. The compound according to claim 16 wherein said compound is encapsulated in a liposome.

24. A composition comprising the compound according to claim 16 and a carrier.
